(19) United States
(12) Patent Application Publication
(10) Pub. No.: US 2012/0071933 A1
(43) Pub. Date: Mar. 22, 2012

(54) RETAINING MECHANISM

(75) Inventor: Steven DeRidder, Bartlett, TN (US)

(73) Assignee: WARSAW OROTHOPEDIC, INC., Warsaw, IN (US)

(21) Appl. No.: 12/887,030

(22) Filed: Sep. 21, 2010

(51) Int. Cl. A61B 17/88; A61B 17/80

(52) U.S. Cl. 606/281; 606/289

(57) ABSTRACT

A retaining mechanism for use in affixing a stratum to bone is disclosed. The retaining mechanism comprises a stratum and a retaining element. The stratum comprises a first surface, a second surface, and at least one hole extending between the first surface and the second surface, wherein the second surface is configured to engage at least a portion of the bone. The retaining element is configured to engage the stratum and configured to at least partially overlap the at least one hole such that the retaining element helps prevent inadvertent backing out of the fastener after the fastener has been fully inserted into the at least at least one hole of the stratum, wherein the retaining element is substantially rigid. Further, methods of implanting a spinal plate are disclosed.

### FIELD OF INVENTION

The present invention is directed to systems for affixing a stratum to bone.

### BACKGROUND

The present disclosure relates to retaining mechanisms, and more particularly, systems for affixing a stratum to bone.

### SUMMARY OF THE INVENTION

A retaining mechanism for use in affixing a stratum to bone is disclosed. The retaining mechanism comprises a stratum and a retaining element. The stratum comprises a first surface, a second surface, and at least one hole extending between the first surface and the second surface, wherein the second surface is configured to engage at least a portion of the bone. The retaining element is configured to engage the stratum and configured to at least partially overlap the at least one hole such that the retaining element helps prevent inadvertent backing out of the fastener after the fastener has been fully inserted into the at least at least one hole of the stratum, wherein the retaining element is substantially rigid.

In other embodiments, a system for affixing a stratum to bone is disclosed. In such embodiments, the system comprises a stratum, a retaining element and a fastener. In such embodiments, the stratum comprises a first surface, a second surface, and at least one hole extending between the first surface and the second surface, wherein the second surface is configured to engage at least a portion of the bone. In such embodiments, the retaining element is configured to engage the stratum and configured to at least partially overlap the head of the fastener such that the retaining element helps prevent inadvertent backing out of the fastener after the fastener has been fully inserted into the at least one hole of the stratum, wherein the retaining element is substantially rigid. In such embodiments, the fastener is configured to pass at least partially through the at least one hole and configured to engage the at least a portion of the bone, wherein the fastener further comprises a head portion configured for manipulation by a user and a shaft portion configured to engage at least a portion of bone.

Further, methods of implanting a spinal plate are disclosed.

### BRIEF DESCRIPTION OF THE DRAWINGS

FIG. 1 is a schematic, cross-sectional view of two adjacent vertebral bodies;

FIG. 2 is an isometric view of a system for affixing a stratum to bone;

FIG. 3 is an isometric view of the retaining mechanism of the system of FIG. 2;

FIG. 4 is an isometric view of the retaining element of the retaining mechanism of FIG. 3;

FIG. 5 is a front view of system of FIG. 2 in cooperation with vertebral bodies; and

FIG. 6 is a cross-sectional, side view of the system of FIG. 2 in cooperation with vertebral bodies.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

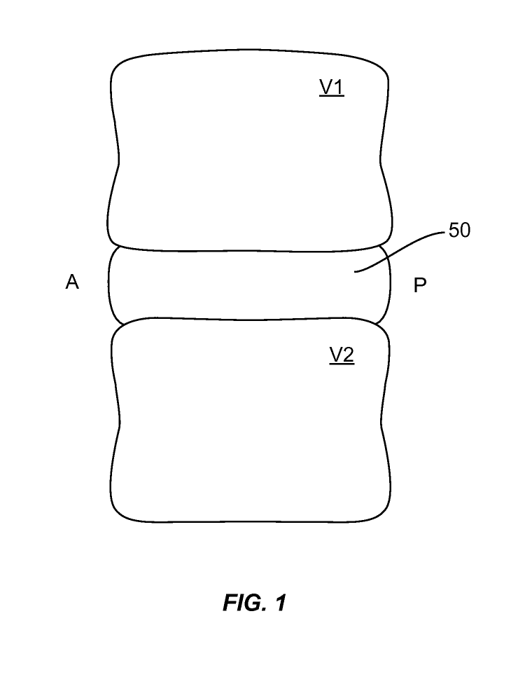

FIG. 1 shows a schematic, cross-sectional view of two adjacent vertebral bodies V1 and V2 with an intervertebral disc 50 situated in its natural location between the two vertebral bodies V1 and V2. As shown in FIG. 1, vertebral body V1 represents a superior vertebral body and V2 represents an inferior vertebral body. Reference marker A represents an anterior side of the vertebral bodies V1 and V2, whereas reference marker P represents a posterior side of the vertebral bodies V1 and V2.

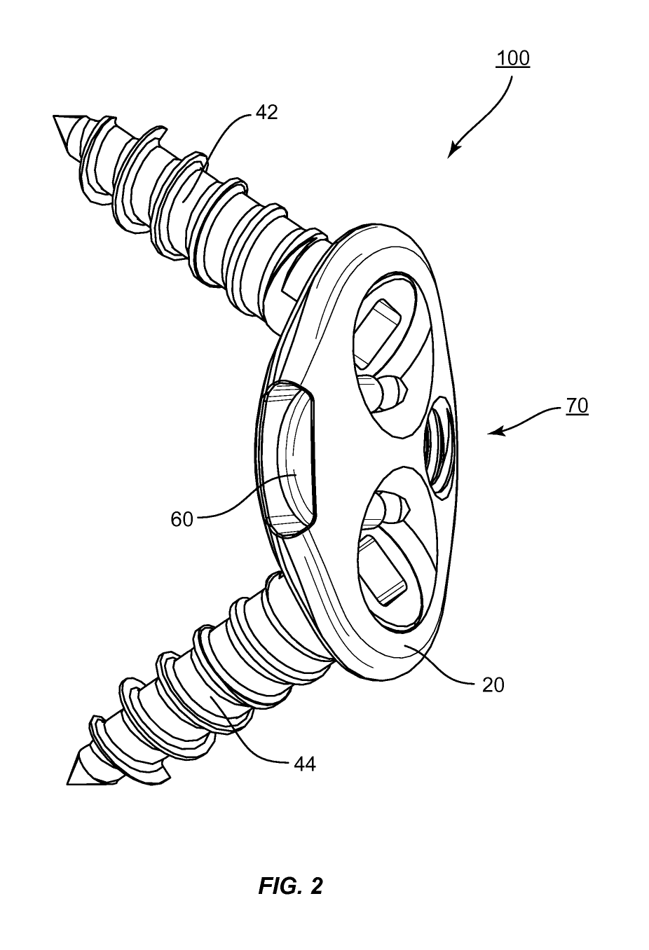

FIG. 2 shows an isometric view of a system 100 for affixing a stratum 20 to bone, for example, V1 and/or V2. The system 100 comprises a retaining mechanism 70 that comprises a stratum 20 and a retaining element 60. Further, as shown in FIG. 2, the system 100 further comprises fasteners 42 and 44.

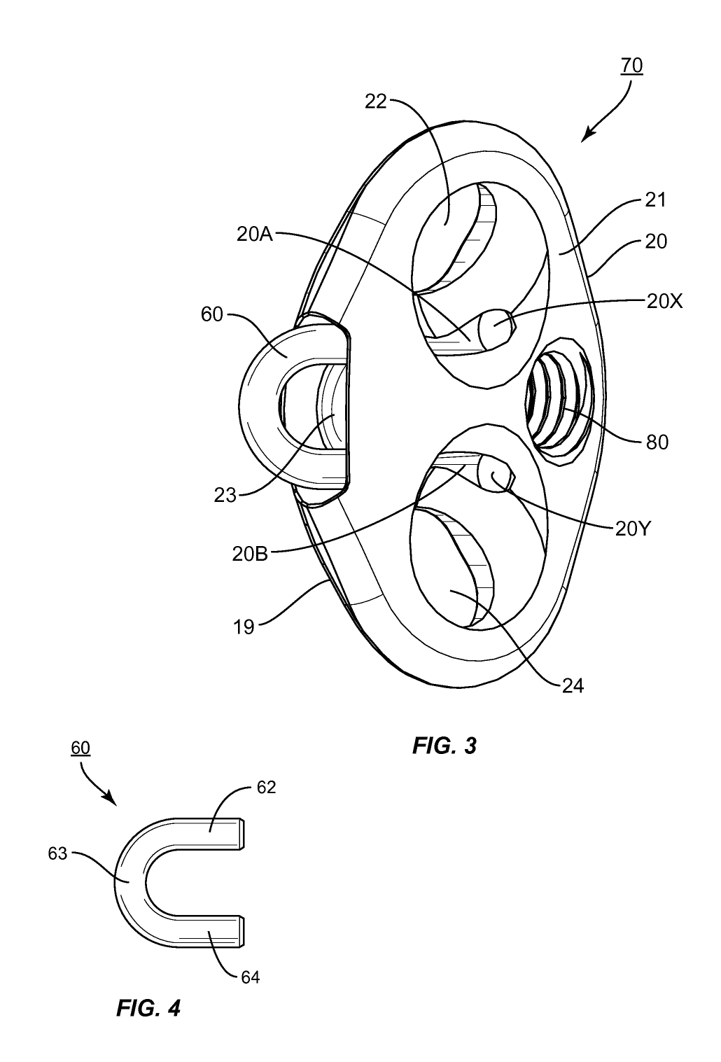

FIG. 3 shows an isometric view of the retaining mechanism 70 of FIG. 2. As shown, the stratum 20 comprises a first surface 21, a second surface 19, and at least one hole 22 or 24 extending between the first surface 21 and the second surface 19, wherein the second surface 19 is configured to engage at least a portion of the bone, for example, V1 and/or V2. As shown in FIG. 3, the retaining mechanism 70 further comprises a retaining element 60 configured to engage the stratum 20 and configured to at least partially overlap the at least one hole 22 or 24 such that the retaining element 60 helps prevent inadvertent backing out of the fastener 42 or 44 after the fastener 42 or 44 has been fully inserted into the at least at least one hole 22 or 24 of the stratum 20, wherein the retaining element 60 is substantially rigid. Note that as shown in FIG. 3, the retaining element 60 is engaged with the stratum 20, but has not been moved to its fully-inserted position where it at least partially overlaps the at least one hole 22 or 24. As shown in FIG. 3, the stratum 20 further comprises a hole 80 for engaging an instrument for holding and/or inserting the stratum 20 in place.

The term “substantially” as used herein may be applied to modify any quantitative representation which could permissibly vary without resulting in a change in the basic function to which it is related. For example, a retaining element 60 may be considered substantially rigid if when it is in its fully-inserted position and at least partially overlapping a hole, for example, 22 or 24, it does not deflect enough to allow a fastener, for example, 42 or 44, to inadvertently back out of the stratum 20.

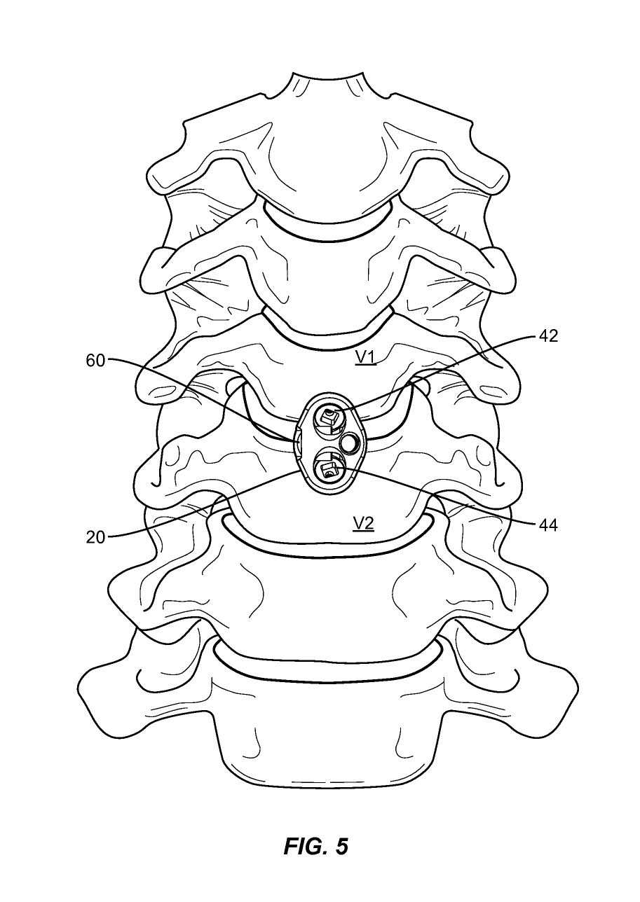

FIG. 4 shows an isometric view of the retaining element 60 of the retaining mechanism 70 of FIG. 3. As shown in FIG. 4, the retaining element 60 comprises a first leg 62, a second leg 64 and a junction 63 between the two legs 62 and 64. As shown in FIG. 4, the retaining element 60 is U-shaped. As shown in FIG. 3, the stratum 20 has a first groove 20A, a second groove 20B and a recess 23 in between grooves 20A and 20B for receiving and engaging the retaining element 60. Specifically, the first groove 20A engages the first leg 62 of retaining element 60 and the second groove 20B engages the second leg 64 of retaining element 60. Further, as shown in FIGS. 3 and 4, the first groove 20A has an end 20× and the second groove 20B has an end 20Y. As shown in FIG. 3, when the retaining element 60 is in its fully-inserted position, the ends of the first and second legs 62 and 64 of the retaining element 60 do not engage ends 20X and 20Y, respectively. In addition, as shown in FIGS. 2 and 3, the stratum 20 can accommodate a first fastener 42 in the first hole 22 and can accommodate a second fastener 44 in the second hole 24. Accordingly, as shown in FIGS. 2-4, the retaining element 60 is configured to at least partially overlap the first hole 22 and to at least partially overlap the second hole 24.

As shown in FIGS. 3 and 4, the stratum 20 is further configured for temporarily locking the retaining element 60 in its fully-inserted position. For example, the legs 62 and 64 of the retaining element 60 may have for example, an interference fit with the grooves 20A and 20B, respectively. The engagement between the stratum 20 and the retaining element 60 need not be one that achieves a temporary lock. It may be a permanent or semi-permanent lock. That is, for example, when the lock is temporary, with the use of

sufficient force, the retaining element 60 may be removed from the stratum 20 by pulling it out of the stratum 20 in the direction opposite that from which it was inserted into the stratum 20. When the need for removal of retaining element 60 arises, one way is to apply pressure to the legs 62 and 64 in the direction opposite from which it was inserted and away from surfaces 20X and 20Y, respectively. As one would be separating the retaining element 60 from the stratum 20, the stratum 20 or portion thereof may be used as a base from which pressure may be applied to the legs 62 and 64, for example, by prying the legs 62 and 64 away from the stratum 20.

Although the retaining element 60 is shown as having a U-shape, the retaining element 60 may have different shapes, for example a substantially straight length of material such as a rod. That is, the retaining element 60 may take any form that satisfies its function described herein, for example, being able to adequately engage with the stratum 20 and being substantially rigid so that when it is in its fully-inserted position and at least partially overlapping a hole 22 or 24, it does not deflect enough to allow the fastener 42 or 44 to inadvertently back out of the stratum 20.

FIG. 5 shows a front view of system 100 of FIG. 2 in cooperation with vertebral bodies V1 and V2. As shown in FIG. 5, fastener 42 passes through hole 22 of stratum 20 and engages vertebral body V1, and fastener 44 passes through hole 24 of stratum 20 and engages vertebral body V2. Further, as shown in FIG. 5, the fasteners 42 and 44 are in their fully-inserted positions and the retaining element 60 is in its fully-inserted position such that the retaining element 60 helps prevent inadvertent backing out of the fasteners 42 and 44. Specifically, as shown in FIG. 5, each leg 62 and 64 of the retaining element 60 at least partially overlaps the respective holes 22 and 24 and also at least partially overlaps the respective heads 42H and 44H of fasteners 42 and 44.

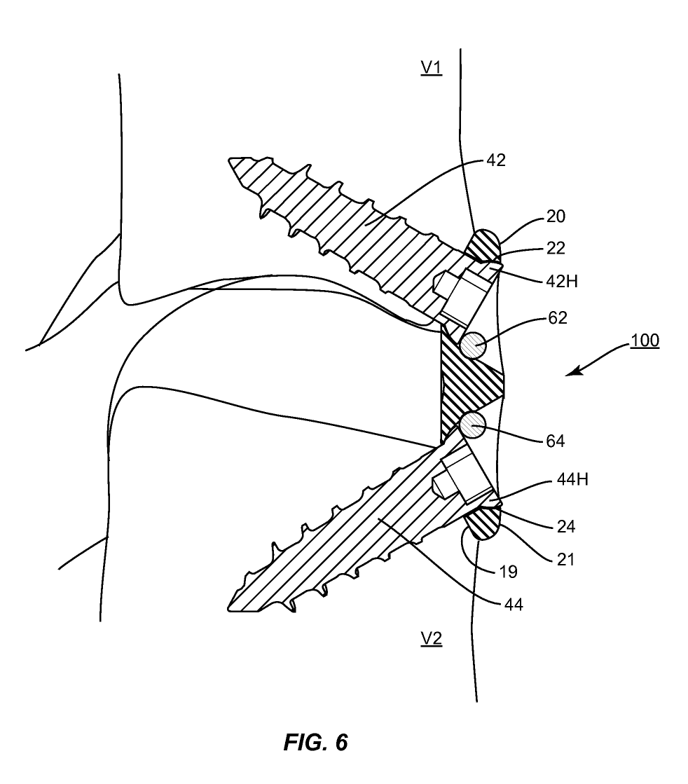

FIG. 6 shows a cross-sectional, side view of the system 100 in cooperation with vertebral bodies V1 and V2. FIG. 6 shows shaft portions (not labeled) and head portions 42H and 44H of fasteners 42 and 44, respectively. Each shaft portion is configured for engaging at least a portion of the vertebral body V1 or V2, and each head portion is configured for manipulation by a user, for example, with the aid of an instrument. As shown in FIG. 6, the fasteners 42 and 44 are in their fully-inserted positions and the retaining

element 60 is in its fully-inserted position such that the retaining element 60 helps prevent inadvertent backing out of the fasteners 42 and 44. Specifically, as shown in FIG. 6, each leg 62 and 64 of the retaining element 60 at least partially overlaps the respective holes 22 and 24 in the stratum 20 and also at least partially overlaps the respective heads 42H and 44H of fasteners 42 and 44.

Although the retaining element 60 is shown as at least partially overlapping the heads 42H and 44H of fasteners 42 and 44, the retaining element 60 may overlap a portion of the fastener or fasteners 42 and 44 other than the heads 42H and 44H. For example, the retaining element 60 may at least partially overlap a portion of a fastener or fasteners 42 and 44 situated lower (or more distal along the fastener) than the heads. In such case, for example, the retaining element 60 may engage a portion of the head 42H and 44H, for example, grooves (not shown) in the heads 42H and 44H provided to engage with the retaining element 60, to thereby help prevent inadvertent backing out of the fastener or fasteners 42 and 44 after the fastener or fasteners 42 or 44 have been fully inserted into the holes 22 and 24 of the stratum 20.

As shown in the Figures and as described herein, the bone may be, for example, part of a spine such as a vertebral body or vertebral bodies, the stratum 20 may be, for example, a spinal plate, and the fastener may be, for example, a screw.

Implantation of a spinal plate 20 may, for example, comprise the following steps: (1) positioning a retaining element 60 in the spinal plate 20 so that it is engaged with the spinal plate 20, but that the retaining element 60 is not in its fully-inserted position so that the retaining element 60 does not block insertion of the fasteners 42 and 44 into the holes 22 and 24 on the plate 20, for example, as shown in FIG. 3; (2) placing the spinal plate 20 (along with the retaining element) in a desired location on a spine; (3) inserting the fasteners 42 and 44 into the holes 22 and 24 until it is in a desired position (for example, their fully-inserted positions); and (4) moving the retaining element 60 on the spinal plate 20 such that the retaining element 60 at least partially overlapping the heads 42H and 44H of the fasteners 42 and 44, as shown in FIG. 5, thereby helping prevent inadvertent backing out of the fasteners 42 and 44.

In the embodiments described here, the stratum 20 may be made of a variety of biocompatible materials (metal or non-metal), including but not limited to, Titanium Alloys, commercially available Titanium, stainless steel, polyetheretherketone (“PEEK”), cobalt chrome (“CoCr”), polyetherketoneketone (“PEKK”), ultra high molecular weight polyethylene (“UHMWPE”), polyethylene, shape memory metals, other polymers or any combination of such materials. Similarly, the retaining element 60 and/or the fasteners 42 and 44 may be made of the same materials. Also, any suitable materials know in the art may work for each of these elements.

Whatever material or materials are used to make the retaining element 60, the retaining element 60 described herein is substantially rigid so that when it is in its fully-inserted position and at least partially overlapping a hole 22 or 24 and/or head 42H or 44H of a fastener 42 or 44, it does not deflect enough to allow the fastener 42 or 44 to inadvertently back out of the stratum 20. In this regard, for example, the retaining element 60 may consist essentially of PEEK or may consist essentially of metal.

All adjustments and alternatives described above are intended to be included within the scope of the invention, as defined exclusively in the following claims. Those skilled in the art also should realize that such modifications and equivalent constructions or methods do not depart from the spirit and scope of the present disclosure, and that they may make various changes, substitutions, and alterations herein without departing from the spirit and scope of the present disclosure. Furthermore, as used herein, the terms components and modules may be interchanged. It is understood that all spatial references, such as “superior,” “inferior,” “anterior,” “posterior,” “outer,” “inner,” and “perimeter” are for illustrative purposes only and can be varied within the scope of the disclosure.

1. A retaining mechanism for use in affixing a stratum to bone, the mechanism comprising:
a stratum comprising a first surface, a second surface, and at least one hole extending between the first surface and the second surface, wherein the second surface is configured to engage at least a portion of the bone, and
a retaining element configured to engage the stratum and configured to at least partially overlap the at least one hole such that the retaining element helps prevent inadvertent backing out of the fastener after the fastener has been fully inserted into the at least at least one hole of the stratum, wherein the retaining element is substantially rigid.

2. The mechanism of claim 1, wherein the stratum is further configured to engage the retaining element.

3. The mechanism of claim 1, wherein the stratum is further configured for temporarily locking the retaining element in its fully-inserted position.

4. The mechanism of claim 1, wherein the bone is spine, and wherein the stratum is a spinal plate.

5. A system for affixing the stratum of claim 1 to the bone, the system comprising:
the retaining mechanism; and
a fastener configured for passing through the hole in the stratum and engaging the bone.

6. The mechanism of claim 1, wherein the retaining element is U-shaped.

7. The mechanism of claim 6 further comprising a second hole between the first surface and the second surface, wherein the retaining element is configured to at least partially overlap the first hole and to at least partially overlap the second hole.

8. The mechanism of claim 1, wherein the retaining element consists essentially of polyetheretherketone.

9. The mechanism of claim 1, wherein the retaining element consists essentially of metal.

10. The system of claim 5, wherein the fastener is a screw.

11. A system for affixing a stratum to bone, the system comprising:
a stratum having a first surface, a second surface, and at least one hole extending between the first surface and the second surface, wherein the second surface is configured to engage at least a portion of the bone;
a fastener configured to pass at least partially through the at least one hole and configured to engage the at least a portion of the bone, the fastener further comprising:
a head portion configured for manipulation by a user; and
a shaft portion configured to engage at least a portion of bone; and
a retaining element configured to engage the stratum and configured to at least partially overlap the head of the fastener such that the retaining element helps prevent inadvertent backing out of the fastener after the fastener has been fully inserted into the at least one hole of the stratum, wherein the retaining element is substantially rigid.

12. The system of claim 11, wherein the stratum is further configured to engage the retaining element.

13. The system of claim 11, wherein the stratum is further configured to temporarily lock the retaining element in its fully-inserted position.

14. The system of claim 11, wherein the bone is spine, wherein the stratum is a spinal plate, and wherein the fastener is a screw.

15. The system of claim 11, wherein the retaining element is U-shaped.

16. The system of claim 11, wherein the stratum further comprises a second hole between the first surface and the second surface, wherein the retaining element is configured to at least partially overlap the first hole and to at least partially overlap the second hole.

17. The system of claim 11, wherein the retaining element consists essentially of polyetheretherketone.

18. The system of claim 11, wherein the retaining element consists essentially of metal.

19. A method of implanting a spinal plate, the method comprising the following steps:
placing the spinal plate in a desired location on a spine, the plate comprising:
a first surface, a second surface, and at least one hole extending between the first surface and the second surface, wherein the second surface is configured to engage at least a portion of the spine, and
a retaining element configured for engaging the spinal plate and configured to at least partially overlap the at least one hole, wherein the retaining element is substantially rigid;
while the retaining element does not block insertion of a fastener into the at least one hole, inserting the fastener into the at least one hole until it is in a desired position; and
moving the retaining element on the spinal plate such that the retaining element helps prevent inadvertent backing out of the fastener.

20. The method of claim 19, wherein the fastener comprises a head portion configured for manipulation by a user, and a shaft portion configured for engaging at least a portion of spine, wherein after the step of moving the retaining element, the retaining element at least partially overlaps the head of the fastener such that the retaining element helps prevent inadvertent backing out of the fastener.
